(19) United States
(12) Patent Application Publication
(10) Pub. No.: US 2003/0049210 A1
(43) Pub. Date: Mar. 13, 2003

(54) Oral composition

(75) Inventor: Matthieu Lecharny, Milan (IT)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/225,847

(22) Filed: Aug. 22, 2002

(30) Foreign Application Priority Data
Sep. 3, 2001 (EP) 01307473.7

(51) Int. Cl. A61K 7/16

(52) U.S. Cl. 424/49

(57) ABSTRACT

An oral hygiene kit comprising an oral care composition for use in a toothbrushing regime and, independently therefrom, a plaque disclosing means, characterised in that the plaque disclosing means is capable of being used before a toothbrushing session such that any plaque present on the surfaces of the teeth can be indicated to the user prior to brushing with the oral care composition.

### BACKGROUND OF THE INVENTION

1. Field of the Invention

The present invention relates to an oral composition and a means for disclosing plaque.

2. The Related Art

Plaque disclosing devices are known in the art. Typically these are in the form of tablets which comprise a suitable stain which is selective to plaque and these are crunched in the oral cavity to stain the plaque. The user is then able to identify whether the teeth are being brushed properly or not.

Disclosing agents for dental plaque as a means of measuring tooth cleanliness and to effect proper oral hygiene practices, have been widely explored in the prior art.

Disclosing agents include organic dyes such as erythrosin (FDC Red #3) as disclosed in U.S. Pat. No. 3,309,274 by Brilliant, and U.S. Pat. No. 3,624,219 by Perlitsch. Block patents U.S. Pat. Nos. 3,723,613; 3,997,658 and 4,064,229 disclose a two-tone dye test comprising the combination of the erythrosin with either FDC Green #3, FDC Blue #1 or Hercules Green Shade 3 in order to obtain differential staining, i.e. thick old plaques stain blue and thin new plaques stain red.

Gaffar, U.S. Pat. No. 4,431,638, discloses a red vegetable dye obtained from sugar beets as a plaque disclosant. Kosti, U.S. Pat. No. 4,348,378 discloses rupturable microencapsulated dyes as a plaque disclosant. Frysh, U.S. Pat. No. 4,666,700 discloses as a plaque disclosant a water insoluble pigment such as a lake.

GB 2 274 061 (King) discloses an oral composition comprising two disctinct components. A first component is a regular oral composition while the second is a plaque disclosing composition. Both components are dispensed from a single package and mixed during use.

Despite the prior art there remains the need for improved hygiene and accordingly it is an object of the present invention to improve the use of oral compositions and to create a more confident feeling in the consumer.

### SUMMARY OF THE INVENTION

Accordingly, the present invention relates to an oral hygiene kit comprising an oral care composition for use in a toothbrushing regime and, independently therefrom, a plaque disclosing means, characterised in that the plaque disclosing means is capable of being used before a toothbrushing session such that any plaque present on the surfaces of the teeth can be indicated to the user prior to brushing with the oral care composition.

The means for disclosing plaque can be any means already available in the prior art. In particular it may be a tablet, mouthwash or gel as well as a device, which acts as an applicator for such. For example, an applicator may comprise a handle and a head, the head comprising a plaque disclosing composition either in latent, dried form or even one which is ready for use without any activation.

The oral composition preferably includes any oral composition form, which is available on the market, e.g. dentifrices, gels, mouthwashes, powders, lozenges, pastilles etc.

The invention lies in the fact that we have surprisingly found that the combination of the two provides an perceivable improvement of the oral composition by the consumer, i.e. there is an increased whitening effect of the oral composition, increased gum health, reduced bad breath, reduced plaque etc. This is in contrast with the prior art where it is usual for a consumer to use a plaque disclosing device only sporadically and not in concert with their usual oral composition. Accordingly, they fail to achieve the benefits of the invention. Further, it is surprising that the consumer using this product perceives improvements in their oral environment which are not directly related to the presence of plaque. For example, consumers using a kit according to the invention experience fresher breath.

The kit according to the invention comprises an oral care composition to be used as part of a daily toothbrushing regime. The plaque disclosing means can be used as many times as possible. For example, should the plaque disclosing means be a number of plaque disclosing tablets obviously each can only be used once. Accordingly, the consumer will be directed to use one tablet per week for example so that the total number of tablets can be used for the expected lifetime of the oral care composition. Typically, the kit will comprise two hermetically sealed plaque disclosing means and preferably these will be tablets.

It is not part of the invention that where the plaque disclosing means is a further composition that the oral care composition and plaque disclosing means are to be used simultaneously. It is difficult to see how the consumer would benefit by brushing with a dentifrice that comprised two distinct formulations: one a regular oral composition and the other a plaque disclosing composition both being dispensed from their packaging simultaneously and mixed during use.

The oral composition according to the invention comprise further ingredients which are common in the art, such as:

antimicrobial agents, e.g. Triclosan, chlorhexidine, copper-, zinc- and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as keratin and collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition.

humectants such as glycerol, sorbitol, propyleneglycol, xylitol, lactitol etc.;

binders and thickeners such as sodium carboxymethyl-cellulose, xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included. Examples of such polymers are copolymers of polyvinylmethylether with maleic anhydride and other similar delivery enhancing polymers, e.g. those described in DE-A-3,942,643 (Colgate);

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

According to a second aspect of the invention there is provided the use of a kit according to the invention for the treatment of bad breath.

In a further aspect there is provided the use of a kit according to the invention for whitening the teeth.

In a final aspect to the invention there is provided the use of a kit according to the invention for improving gum health.

The foregoing description and examples illustrate selected embodiments of the present invention. In light thereof variations and modifications will be suggested to one skilled in the art, all of which are within the spirit and purview of this invention.

### EXAMPLES

The following consumer test indicates the benefits which can only be attributed to the presence of plaque disclosing means with a dentifrice.

A consumer panel was split into two groups. One was given a dentifrice composition while the other was given a kit comprising exactly the same dentifrice composition and two plaque disclosing tablets.

Each consumer group was asked to use the test product for a ten day trial period before being asked to judge on the following characteristics:

1. Whether the product gives complete care to the mouth.

2. Whether it gives complete protection.

3. Whether it takes care of both teeth and gums.

4. Whether it cleans teeth thoroughly.

5. Whether it leaves the mouth feeling fresh for a long time.

6. Whether it helps to keep teeth healthy.

7. Whether it is effective in preventing decay.

8. Whether it is effective against tartar.

9. Whether it fights plaque effectively.

The marks awarded ranged along a scale running from 0 (no) to 5 (agree strongly) and were averaged.

The marks were as follows:

| Question | Kit | Dentifrice alone |
|---|---|---|
| 1 | 4.20 | 4.08 |
| 2 | 4.18 | 4.04 |
| 3 | 4.15 | 4.07 |
| 4 | 4.21 | 4.08 |
| 5 | 4.05 | 3.94 |
| 6 | 4.15 | 4.11 |
| 7 | 4.02 | 3.95 |
| 8 | 4.01 | 3.70 |
| 9 | 4.04 | 3.78 |

What is claimed is:

1. An oral hygiene kit comprising an oral care composition for use in a toothbrushing regime and, independently therefrom, a plaque disclosing means, characterised in that the plaque disclosing means is capable of being used before a toothbrushing session such that any plaque present on the surfaces of the teeth can be indicated to the user prior to brushing with the oral care composition.

2. A kit according to claim 1, wherein the oral composition is a dentifrice.

3. A kit according to claim 1 or 2, wherein the device for disclosing plaque is a tablet.

4. A kit according to claim 1 or 2, wherein the means for disclosing plaque is an applicator comprising a handle and an applicator head, said head comprising a plaque disclosing composition.

5. Use of a kit according to any preceding claim for whitening the teeth.

6. Use of a kit according to any of claims 1 to 4 for improving gum health.

7. Use of a kit according to any of claims 1 to 4 for the treatment of bad breath.

8. An oral hygiene kit comprising a dentifrice and a plurality of hermetically sealed plaque disclosing tablets.
